(19) United States
(12) Patent Application Publication
(10) Pub. No.: US 2009/0270850 A1
(43) Pub. Date: Oct. 29, 2009

(54) DEVICES AND METHODS FOR THE ABLATION OF TISSUE IN THE LATERAL DIRECTION

(75) Inventors: Gan Zhou, Plano, TX (US); Jeffrey R. Gladden, Westlake, TX (US)

Correspondence Address: HITT GAINES P.C., P.O. BOX 832570, RICHARDSON, TX, 75083, US

(73) Assignee: Tea Time Partners, L.P., organized in Texas, Rockwall, TX (US)

(21) Appl. No.: 12/137,664

(22) Filed: Jun. 12, 2008

Related U.S. Application Data
(60) Provisional application No. 60/945,124, filed on Jun. 20, 2007.

(51) Int. Cl. A61B 18/24; A61B 18/18; A61B 8/13; A61M 25/10

(52) U.S. Cl. 606/15; 606/16; 606/33; 600/459; 604/96.01

(57) ABSTRACT

Various devices for ablating tissue in a lateral direction and methods of operation thereof. One embodiment of such a device includes: (1) an elongated body configured to carry ablative energy from an ablative energy source associated with a proximal end to a distal end and (2) a distal tip located at the distal end, the distal tip configured to deliver the ablative energy in a direction substantially lateral to a longitudinal axis of the elongated body.

### CROSS-REFERENCE TO RELATED APPLICATIONS

This application claims the benefit of U.S. Provisional Application Ser. No. 60/945,124, entitled “Devices for Ablating Tissue in the Lateral Direction,” filed by Zhou, et al., commonly assigned with this invention and incorporated herein by reference. This application is also related to U.S. patent application Ser. No. 11/315,546, entitled “Image-Guided Laser Catheter,” filed by Zhou on Dec. 22, 2005 and U.S. patent application Ser. No. 11/927,889, entitled “Ultrasonic Pressure Sensor and Method of Operating the Same,” filed by Zhou on Oct. 30, 2007, commonly assigned with this invention and incorporated herein by reference.

### TECHNICAL FIELD OF THE INVENTION

The invention relates generally to the field of medical catheters and guidewires and more specifically to devices, including catheters and guidewires, and methods for the ablation of tissue in the lateral direction.

### BACKGROUND OF THE INVENTION

In interventional cardiology, catheters and guidewires are often inserted into a patient's artery or vein to help accomplish tasks such as angioplasty or for pacemaker or defibrillator lead insertion. For example, a balloon dilation catheter expands at a site of blood vessel occlusion and compresses the plaque and improves patency of the vessel. An intravascular ultrasound catheter provides a 360° view of the lateral cross section of a vessel. Different types of atherectomy procedures are performed using devices such as the rotablade, laser catheter, radio-frequency (RF) catheter or ultrasonic ablation catheter. The remarkably successful stents are deployed with the help of a balloon catheter.

One disease that remains difficult to treat interventionally is due to the inherent nature of the disease and the lack of adequate tools and devices is chronic total occlusion (CTO). Some of the early devices, such as the Magnum™ guidewire (Schneider, Zurich, Switzerland), were made of a Teflon-coated steel shaft with an olive blunt tip. Results using this device in 800 chronic cases of CTO showed angiographic success in only 64% of the cases.

The Kensey™ catheter (Theratech, Miami, Florida) was a flexible polyurethane catheter with a rotating cam at the distal tip driven by an internal torsion guidewire at a speed of 10,000 rpm. Clinical evaluation in 11 patients with peripheral CTO diseases demonstrated only a 63% successful rate. The development of the device halted due to safety concerns.

The ROTACS™ low speed rotational atherectomy catheter (Oscor, Palm Harbor, Florida) was made of several steel coils connected to a distal blunt tip of 1.9 mm. A motor drove the catheter rotation at 200 rpm. The catheter was unsuccessful due to safety concerns arising from the data that 30% of patients had extensive dissections.

The Excimer Laser Wire™ catheter (Spectranetics, Colorado Springs, Colorado) comprised a bundle of silica fibers that delivered excimer laser energy to the distal tip to ablate atherosclerotic plaque. In one clinical trial, the catheter was found to have a high rate of misalignment and perforation due to a stiff guidewire tip and a lack of guidance.

The Frontrunner™ catheter (LuMend, Redwood City, Calif.) is designed with a blunt tip designed to micro-dissect its way through a CTO. A bilaterally hinged distal tip assembly is manually opened and closed by the clinician to accomplish micro-dissection. The device has found some success in treating peripheral CTOs and also has a niche in treating coronary cases with refractory in-stent CTOs wherein the stent serves to confine and guide the device through the occlusion. However, the Frontrunner™ is not suitable for the majority of coronary CTO cases due to poor steerability and the lack of guidance.

The Safe Cross™ guidewire (Intraluminal Therapeutics, Carlsbad, Calif.) combines RF ablation capability with reflectometry at the distal tip. The optical reflectometry system provides a warning signal when the guidewire tip is too close to the vessel wall, and the RF ablation provides a way to cross hard calcified plaque. The device has had some success in recent clinical trials, but it is difficult to use and has yet to show widespread acceptance by interventionalists. The issue with the Safe Cross™ guidewire is that the optical reflectometry system generates a warning signal so frequently that leaves the operator at a loss as to what to do. Such a “negative” signal only tells the clinician what to avoid and fails to provide positive guidance for guidewire steering and advancement. Furthermore, there is no definitive indication of whether the guidewire tip is intra-luminal or extra-luminal. If for any reason the guidewire tip had accidentally perforated the vessel wall, the reflectometry signal would become useless.

Another way to provide a guidance signal for a catheter is to use laser-induced fluorescence. The healthy tissue of the artery wall and the atherosclerotic plaque attached to the wall have different fluorescent spectra or “signatures.” A system that detects this fluorescent signature should be able to tell whether the distal tip of the catheter is surrounded by healthy tissue or by plaque. A warning signal derived from laser induced fluorescence may have some advantages over the optical reflectometry signal, but the drawbacks are similar, namely, no geometric information about the diseased vessel.

A much more effective intervention method involves the use of imaging to guide the advancement of guidewires and catheters. Fluoroscopy is a well-established real-time external imaging modality. Fluoroscopy is used to guide many procedures, but its efficacy has proven to be rather limited. Even with bi-plane projections, fluoroscopic images are hard to interpret for totally occluded vessel regions. Radiation safety as well as contrast fluid dosage are additional variables that the clinicians must monitor carefully during an already-stressful intervention. Given these considerations, it is clear that an intravascular image-guided device would be highly valuable for intervention procedures.

A plurality of intravascular imaging devices have been developed to date. Angioscopy can supply visual information on the luminal surface, using a fiber bundle to illuminate the intraluminal space and also to collect reflected light to form an image. Angioscopy requires flushing the blood and replacing it with saline, a procedure that requires temporarily occluding the blood vessel and can cause prolonged ischemia to the heart. Because of this problem, angioscopy is used rarely other than for research purposes.

Intravascular ultrasound, or IVUS, can provide a cross-sectional image in a plane perpendicular to the catheter's axis and has become a very successful diagnostic tool in interventional cardiology and other medical applications. IVUS can image through blood with an acceptable range and has become a very successful diagnostic tool in interventional cardiology. In IVUS, an ultrasonic transducer is embedded in the distal end of an imaging catheter. The catheter is advanced through the vascular system to the target area. The transducer emits ultrasonic pulses and listens for echoes from the surrounding tissue to form a one-dimensional image. The catheter can be rotated to obtain two-dimensional imaging data or, alternatively, a solid-state IVUS with an annular array of transducers at the catheter distal surface can be used to perform 2D image scanning. Combined with a controlled pullback motion, the device can also obtain three-dimensional image data in a cylindrical volume centered on the catheter. While IVUS would at first appear to be an attractive solution for guiding the advancement of a guidewire through a CTO, existing IVUS catheters have proven difficult to advance through occluded regions of calcified tissue or tissue having a significant degree of fibrosis. For short occlusions, a clinician might be able to use a forward-looking IVUS to guide the advancement of the guidewire through the blockage, but even such forward-looking IVUS are still under development and not yet commercially available.

Optical coherence tomography is a relatively new imaging modality that has been considered for use in CTO intervention. The module uses low-coherence light interferometry to map out the optical absorption and scattering properties of the tissue under illumination. Optical coherence tomography provides image resolution that is about 10 times better than IVUS, but the imaging range is limited to a maximum of 3 to 4 mm. In addition, imaging through blood is very difficult even with carefully-chosen infrared wavelength for the light source. Without a significantly better imaging range, the microscopic resolution is of little usage to CTO guidance, as the most decisive clue that the clinicians can use during a procedure is the large-scale geometric feature that reveal the contour of the blood vessel wall.

U.S. Pat. No. 4,887,605 by Angelsen, et al., describes a laser catheter with an integrated ultrasound imaging module. A housing at the distal end of the catheter contains the ultrasonic transducer. An optical fiber is placed in a central through bore and delivers laser energy to the tissue to be treated. Unfortunately, this device would be difficult to advance through a CTO, because the area that contains the ultrasonic transducer apparently is bulky and lacks the ability to ablate plaque.

U.S. Pat. No. 4,587,972 by Morantte also described a combined ablation and ultrasound-imaging catheter. The catheter contains a fiber bundle for laser delivery and ultrasound transducers that emits in the forward direction. However, Morantte's catheter is apparently bulky and difficult to advance through CTOs.

Another disease that often involves the use of ablative guidewire or catheter for treatment is lumbar herniated disc. The current discectomy procedure uses ablative devices with the ablative energy delivered at the distal tip, generally in the direction along the device's longitidinal axis. This longitudinal ablative device can sometimes make it difficult for a physician to access the herniated disc and to securely position the ablative device prior to energy delivery, thereby risking damage to nearby nerve roots and other healthy tissues.

All prior art devices, including those described above, deliver ablative energy at the end of their ablative tip, along the forward, or longitudinal, direction.

Unfortunately, this makes them difficult, dangerous or impossible to use if a need exists to ablate tissue lateral to the tip. Such prior art devices offer no guidance information that would give a physician confidence that the correct location is being ablated. Such prior art devices have no suitable way to anchor the ablative tip. What is needed in the art is a device that is more effective at ablating tissue lateral to its tip.

### SUMMARY OF THE INVENTION

To address the above-discussed deficiencies of the prior art, one aspect of the invention provides a device for ablating tissue in a lateral direction. In one embodiment, the device includes: (1) an elongated body configured to carry ablative energy from an ablative energy source associated with a proximal end to a distal end and (2) a distal tip located at the distal end, the distal tip configured to deliver the ablative energy in a direction substantially lateral to a longitudinal axis of the elongated body. In one embodiment, the elongated body is a catheter while in another it is a guidewire for a catheter. Described is an embodiment that provides for the elongated body to be associated with at least one optical fiber with laser light being used to furnish ablative energy. In another embodiment, a plurality of optical fibers is used. Certain embodiments of the invention provide for the ablative energy to be reflected in a lateral direction by an angular surface of a glass element in the distal tip. In another embodiment, the end of the optical fiber (or fibers) terminating in the distal end is polished to an acute angle.

Several other embodiments of the invention are disclosed herein. One such embodiment provides for RF ablative energy to be directed substantially lateral to the longitudinal axis of the elongated body through a port on the distal end. Another particularly useful embodiment provides for a balloon to be located proximate the distal tip for anchoring the elongated body in position. In still another embodiment, a radiopaque marker band is located proximate the distal tip. In yet still another embodiment, an imaging component is located proximate the distal tip. One such embodiment provides for the imaging component to be an intravascular ultrasound device.

The foregoing has outlined certain aspects and embodiments of the invention so that those skilled in the pertinent art may better understand the detailed description of the invention that follows. Additional aspects and embodiments will be described hereinafter that form the subject of the claims of the invention. Those skilled in the pertinent art should appreciate that they can readily use the disclosed aspects and embodiments as a basis for designing or modifying other structures for carrying out the same purposes of the invention. Those skilled in the pertinent art should also realize that such equivalent constructions do not depart from the scope of the invention.

### BRIEF DESCRIPTION OF THE DRAWINGS

For a more complete understanding of the invention, reference is now made to the following descriptions taken in conjunction with the accompanying drawings, in which:

FIGS. 1A and 1B are isometric views of embodiments constructed according to the principles of the invention of a device for tissue ablation in a lateral direction;

FIG. 2 is an isometric view of a distal tip of the device of FIG. 1A, including a port for the delivery of ablative energy in a lateral direction;

FIGS. 3A and 3B illustrate an embodiment of a distal tip of a device constructed in accordance with the invention for the distribution of ablative energy in a lateral direction;

FIG. 4 illustrates an embodiment of a distal tip of a device constructed in accordance with the invention that provides for a bundle of optical fibers terminating near the distal end of the elongated body;

FIG. 5 illustrates an optical fiber located in the bundle of FIG. 4, showing that the end is polished at an acute angle, such as about 50°;

FIG. 6 illustrates a distal tip of an embodiment of a device constructed in accordance with the invention where the ablative energy is RF;

FIG. 7 illustrates an embodiment of the invention described herein for temporarily anchoring an ablative device;

FIG. 8 illustrates an embodiment constructed in accordance with the invention with a “C” shaped radiopaque marker band attached on the side of the elongated body at the distal tip;

FIG. 9 illustrates an embodiment of the invention that includes an imaging component; and

FIG. 10 illustrates a block diagram of an embodiment of a method of ablation of tissue in the lateral direction carried out according to the principles of the invention.

### DETAILED DESCRIPTION

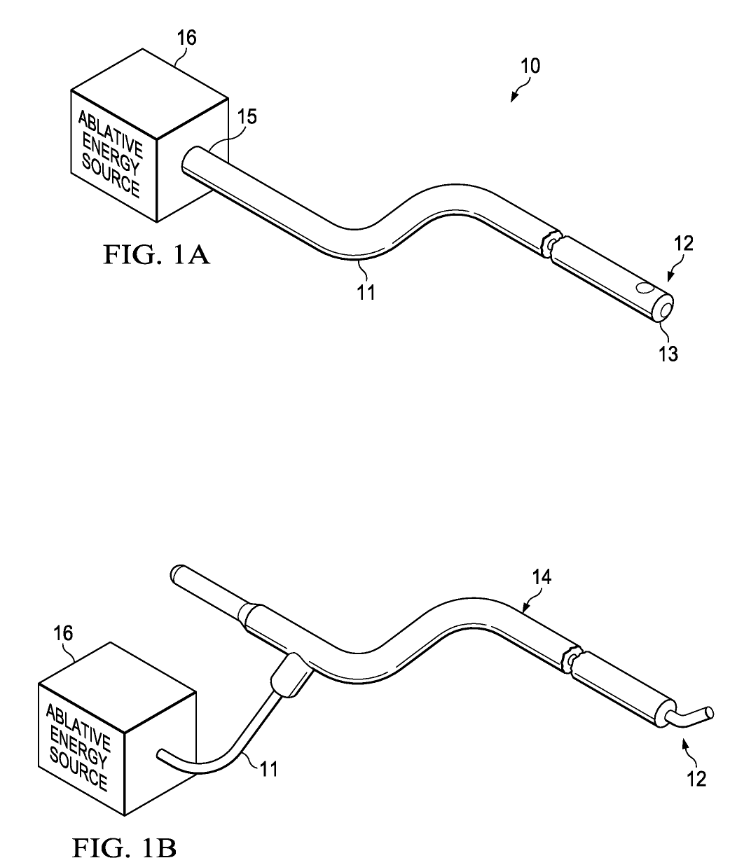

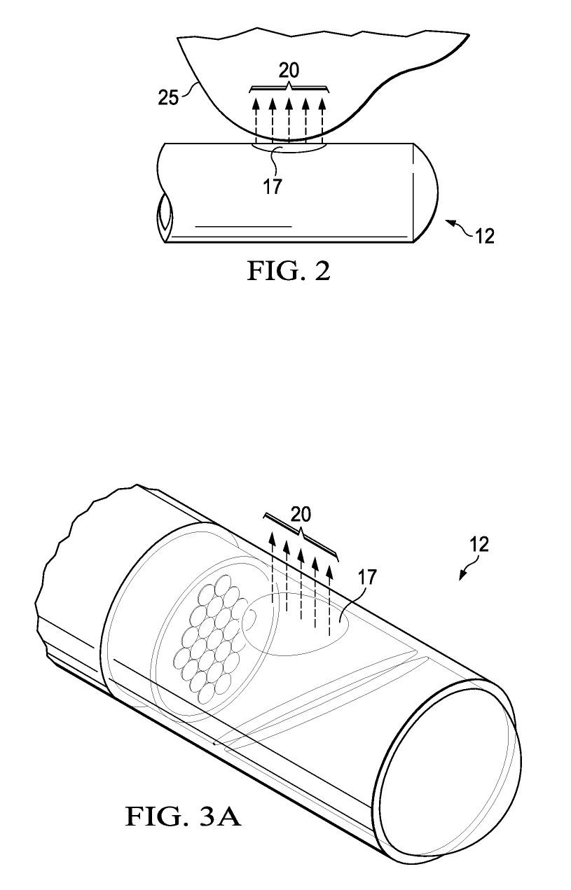

Referring initially to FIGS. 1A and 1B, illustrated are isometric views of embodiments of a device 10 for tissue ablation in a lateral direction that are constructed according to the principles of the invention. FIG. 1A shows an elongated body 11 that is configured to carry ablative energy to a distal end 13 from an ablative energy source 16, such as a console, associated with a proximal end 15. As will be explained with reference to FIG. 2, a distal tip 12 located at the distal end 13 is configured to deliver ablative energy in a direction substantially lateral to a longitudinal axis of the elongated body 11.

The elongated body 11 illustrated in FIG. 1A is representative of a catheter that would typically be inserted by an attending physician into a patient's artery, vein, body cavity or vessel. The embodiment of the invention illustrated in FIG. 1B shows an elongated body 11 that is a guidewire. As is the case with the catheter, the guidewire is associated with a source of ablative energy 16 at its proximal end, which ablative energy is delivered by the distal tip 12 of the guidewire in a direction substantially lateral to the longitudinal axis of the elongated body 11. The guidewire 11 can optionally work in conjunction with a catheter 14. Generally speaking, a guidewire is a wire-like device that is also used to access body cavities and vessels, and sometimes to deliver energy and perform other functions to specific areas of the body. The catheter can also be viewed as a tubular device used for similar applications, except it has a lumen therein to allow fluid passage or to allow other devices, such as a guidewire, to pass through.

Turning now to FIG. 2, illustrated is an isometric view of a distal tip 12 of the device 10 illustrated in FIG. 1A, including a port 17 for the delivery of ablative energy 20 in a lateral direction. Ablative energy 20 carried by the device 10 exits the distal tip 12 in a lateral direction, the angle of which may vary depending on the application, but will in most cases be about perpendicular to the longitudinal axis of the device 10. As illustrated, the ablative energy 20 is used to ablate tissue 25 targeted by the attending physician.

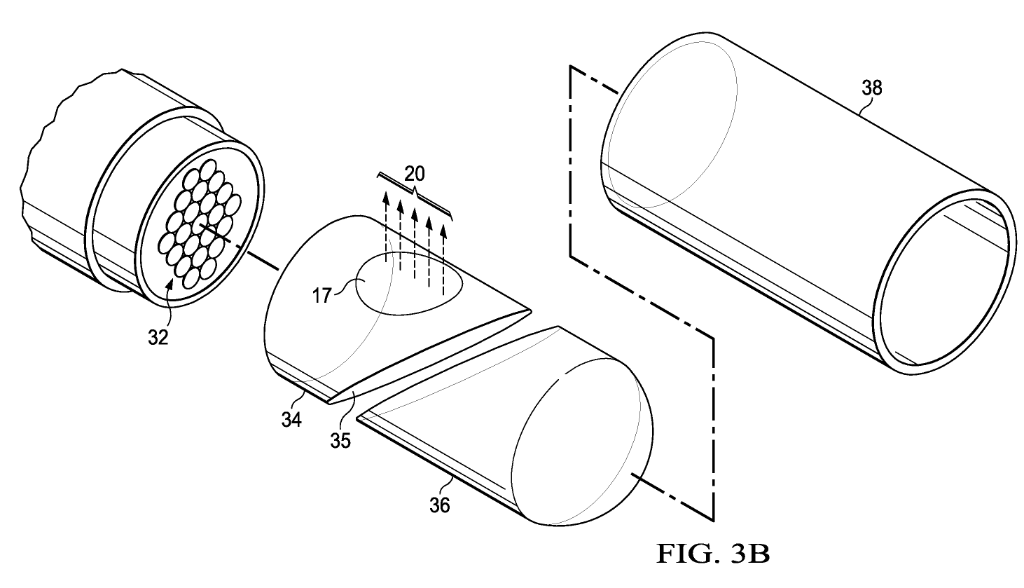

Turning now to FIGS. 3A and 3B, illustrated is an embodiment of a distal tip 12 of a device 10 constructed in accordance with the invention for the distribution of ablative energy 20 in a lateral direction. FIG. 3A shows an embodiment of an assembled distal tip 12 while FIG. 3B shows an exploded view of the distal tip 12 illustrated in FIG. 3A. The illustrated embodiment uses a plurality of optical fibers 32 bundled that carry ablative energy 20 to the distal tip 12. The plurality of optical fibers 32 carry ablative energy 20 introduced into a proximal end 15 of each fiber 32 by an ablative energy source 16, such as a pulsed excimer laser. The bundle of optical fibers 32 is terminated and polished near the distal end 13. The laser ablative energy 20 exits the bundle of optical fibers 32 and enters a glass element 34 that is cut and polished at an angle to form a wedged surface 35. The wedged surface reflects the ablative energy 20 in a direction lateral to the longitudinal axis of the elongated body 11. The glass element 34 is fitted inside a glass collar 38 attached to the bundle of optical fibers 32. The area on the side of the glass collar 30 through which reflected ablative energy 20 passes is an ablative port 17. In some embodiments of the invention, the ablative port 17 may only be an area through which ablative energy 20 passes and may not be distinguished by an opening or similar distinguishing features readily recognize as a “port.” Of course, other embodiments of the invention may have physical characteristics that are readily apparent as being an ablative port 17. The laser generated ablative energy 20 exits the ablative port 17 into the targeted tissue 25 or material. The illustrated embodiment also provides for a plug 36 inside the glass collar 38 that seals and maintains an air gap next to the wedged surface 35, as well as providing a non-traumatic tip to the device 10.

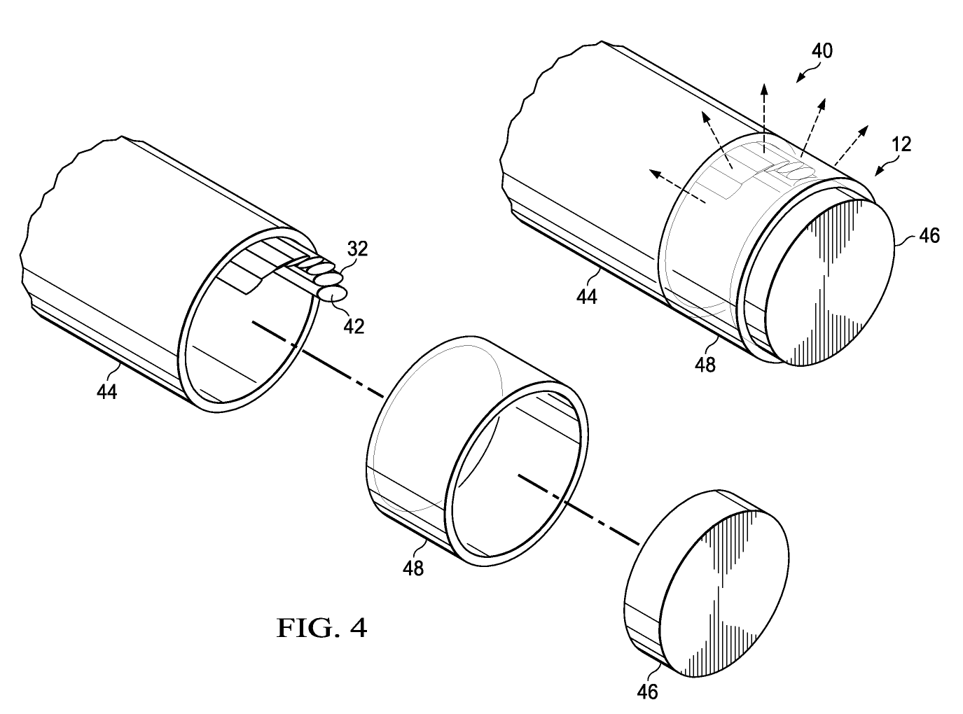

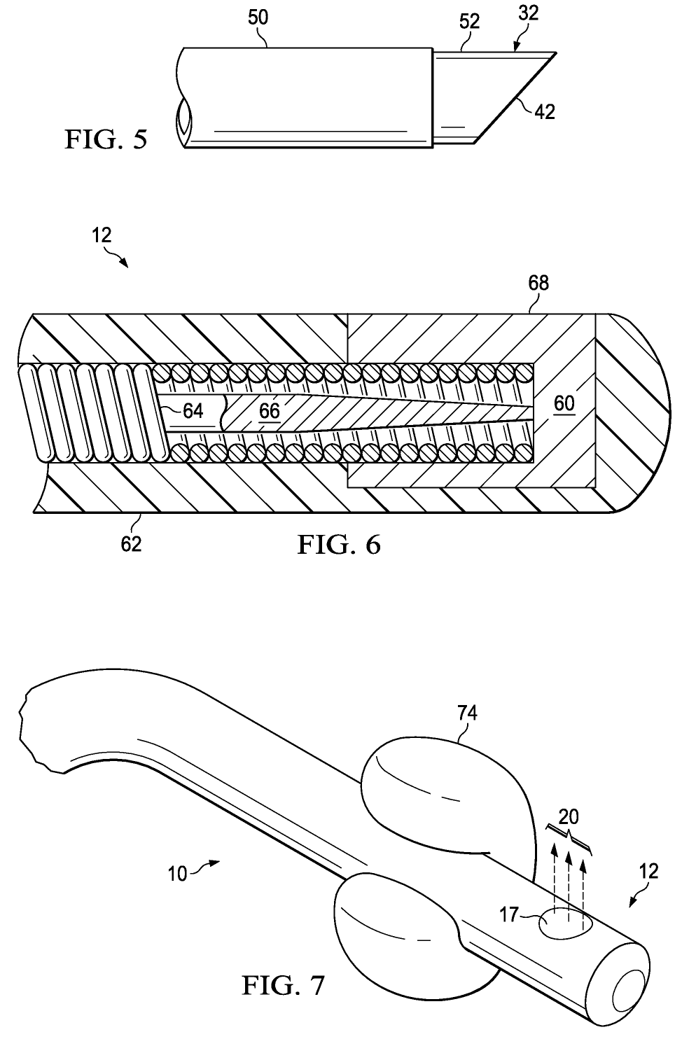

FIG. 4 illustrates another embodiment of a distal tip 12 of a device 10 constructed in accordance with the invention that also uses a bundle of optical fibers 32 terminating near the distal end 13 of the elongated body 11. The distal end 42 of each optical fiber 32 is polished at an acute angle and oriented so that light reflected by such polished distal end 42 surface propagates out of the optical fiber 32 and into the surrounding region substantially lateral to the longitudinal axis of the optical fiber 32. A glass collar 48 and end plug 46 helps seal the region next to each distal end 42 and helps provide for a non-traumatic distal tip 12 to the device 10. FIG. 5 illustrates an optical fiber 32 located in the bundle illustrated in FIG. 4 and shows that the end 42 is polished at an acute angle, such as about 50°. The buffer coating 50 of the optical fiber 32 is stripped near the tip to expose the bare glass 52.

FIG. 6 illustrates a distal tip 12 of an embodiment of a device 10 constructed in accordance with the invention where ablative energy 20 is radio-frequency (RF) energy. Shown in the elongated body 11 of the device 10 is an associated wound coil 64 terminating at an electrode 60 in the distal tip 12. The electrode 60 is partially embedded in insulating material 62 with a small area 68 on the side of the distal tip 12 exposed to the surrounding area that defines an RF ablative port 68. In one embodiment, the coil 64 is stainless steel. In some embodiments, the electrode 60 is an alloy of platinum and cadmium. As shown, a tapered metal safety wire 66 can also be incorporated in the device 10 for structural integrity reasons. As the RF-originated ablative energy exits the device from the ablative port 68, tissue is ablated in a direction substantially lateral to the longitudinal axis of the elongated body 11.

As described in related patent application Ser. No. 11/315,546 by Zhou, entitled “Image Guided Laser Catheter,” and patent application Ser. No. 11/739,301 by Zhou, entitled “Devices and Methods for Ultrasonic Imaging and Ablation,” both of which are commonly assigned with this application and incorporated herein by reference, an ablative device, of the type described herein, is advanced through a body cavity or vessel proximal to the tissue to be ablated. For a number of reasons, in certain applications the attending physician may deem it desirable to temporarily anchor the ablative device. For example, the physician may want to preposition the ablative device while performing other related procedures or it may be necessary to anchor the device while the ablative procedure is performed. Illustrated in FIG. 7 is an embodiment of the invention described herein that can be used to temporarily anchoring an ablative device 10. Shown on the distal tip 12 of the device 10 is an ablative port 17 for the lateral delivery of ablative energy 20. Near the ablative port 17 is a balloon 74 that can be inflated by fluid injection via a lumen (not shown) in the device 10. When inflated the balloon 74 is used to temporarily anchor the device.

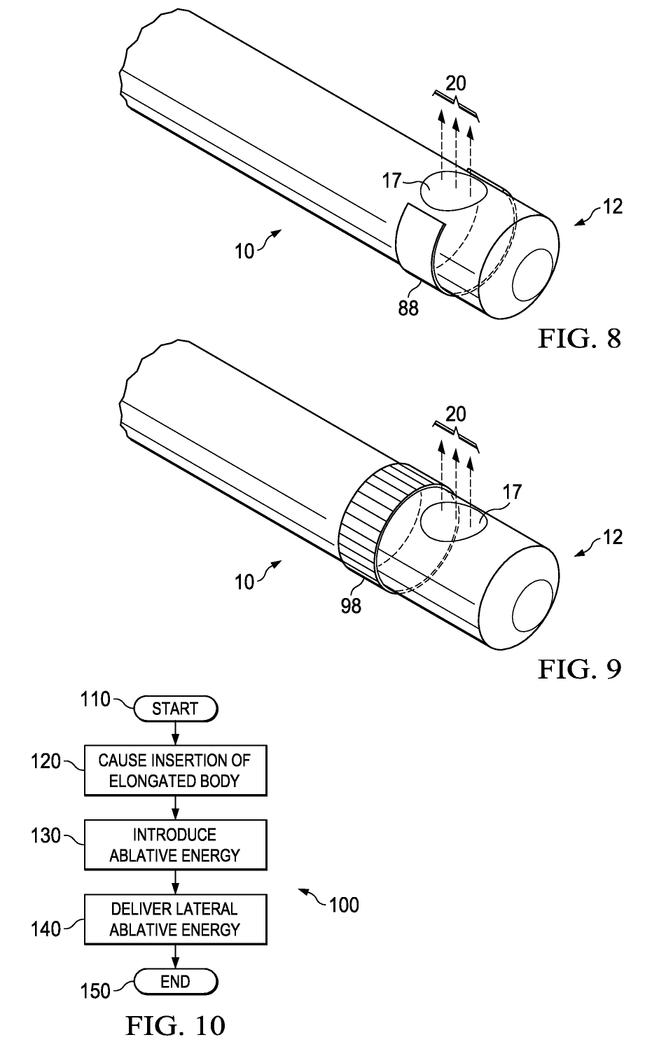

Turning now to FIG. 8, illustrated is an embodiment constructed in accordance with the invention showing a “C” shaped radiopaque marker band 88 attached on the side of the elongated body 11 at the distal tip 12. Ablative energy 20 is delivered to surrounding tissue 25 via an ablative port 17 on the side of the device 10 near the distal tip 12. The illustrated distal tip 12 shows the opening of the “C” shaped radiopaque maker as the ablative port 17 for the delivery of ablative energy 20. By using fluoroscopy methods known to those skilled in the pertinent art, this marker band may be used by a physician to help guide lateral ablative surgery by identifying the lateral region to be ablated.

Turning now to FIG. 9, illustrated is an embodiment of the invention that includes an imaging component 98. This embodiment is usefully employed by a physician to help guide lateral ablative surgery. An ablative port 17 in the distal tip 12 delivers ablative energy 20 in a substantially lateral direction. Shown in close proximity to the ablative port 17 is an imaging component 98. The imaging component 98 can be, for example, an IVUS that performs ultrasonic imaging in a plane about perpendicular to the longitudinal axis of the device 10. Such IVUS devices are widely used in medical procedures and are fabricated from either piezoelectric polycrystals (e.g., lead zirconate titanate, or PZT) or polymers (e.g., polyvinylidene fluoride, or PVDF). Real-time images near the ablative port 17 in the lateral direction can help physicians pinpoint a direction to apply ablative energy 20, thus assuring the region being ablated is the correct region and, thereby, avoid potential damage to healthy tissues. The result is improved efficacy for the surgery as well as enhanced safety.

Turning now to FIG. 10, illustrated is a block diagram of an embodiment of a method of ablation of tissue in the lateral direction 100 carried out according to the principles of the invention. The method commences with a start step 110. In a step 120 an elongated body configured to carry ablative energy to its distal from an ablative energy source associated with its proximal end is inserted into a body cavity or vessel. As will be understood by those skilled in the pertinent art, the elongated body will positioned next to tissue requiring ablation. The elongated body may either be a catheter or it may be an optical fiber within a catheter. In a step 130, an ablative energy source is caused to introduce ablative energy into the elongated body at its proximal end. In a step 140, the distal tip located at the distal end of the elongated body is caused to deliver the ablative energy in a direction substantially lateral to a longitudinal axis of the elongated body. The method 100 concludes with an end step 150.

Those skilled in the art to which the invention relates will appreciate that other and further additions, deletions, substitutions and modifications may be made to the described embodiments without departing from the scope of the invention. It is to be also understood that the invention may be embodied in various forms. Therefore, specific details disclosed herein are not to be interpreted as limiting, but rather as a basis for the claims and as a representative basis for teaching one skilled in the art to employ the invention in virtually any appropriately detailed system, structure or manner.

What is claimed is:

1. A device for ablating tissue in a lateral direction, comprising:
an elongated body configured to carry ablative energy from an ablative energy source associated with a proximal end to a distal end; and
a distal tip located at said distal end, said distal tip configured to deliver said ablative energy in a direction substantially lateral to a longitudinal axis of said elongated body.

2. The device as recited in claim 1 wherein said elongated body is a catheter.

3. The device as recited in claim 1 wherein said elongated body is a guidewire.

4. The device as recited in claim 1 wherein said elongated body is associated with at least one optical fiber and said ablative energy is a laser light.

5. The device as recited in claim 4 wherein said distal tip has an associated glass element with an angular surface to reflect said ablative energy in a direction substantially lateral to said longitudinal axis of said elongated body.

6. The device as recited in claim 5 wherein said glass element is fitted within a collar and said ablative energy is reflected by an angular surface thru a port.

7. The device as recited in claim 1 wherein said elongated body is associated with a plurality of optical fibers.

8. The device as recited in claim 7 wherein said plurality of optical fibers terminate at said distal tip and each is polished at an acute angle and oriented to propagate said ablative energy in a direction substantially lateral to said longitudinal axis of said elongated body.

9. The device as recited in claim 1 wherein said ablative energy is provided by radio frequency (RF) and said elongated body has an associated wound coil terminating at an electrode in said distal tip, wherein said ablative energy is directed substantially lateral to said longitudinal axis of said elongated body through an RF ablative port.

10. The device as recited in claim 9 wherein said coil is made of stainless steel and said electrode is an alloy of either platinum or cadmium.

11. The device as recited in claim 1 further comprising a balloon capable of being inflated by an infusion of liquid injected via a lumen within said elongated body, said balloon located proximate said distal tip.

12. The device as recited in claim 1 further comprising a radiopaque marker band proximate said distal tip.

13. The device as recited in claim 1 further comprising an imaging component proximate said distal tip.

14. The device as recited in claim 13 wherein said imaging component is an intravascular ultrasound device.

15. A method of ablating tissue in a lateral direction, comprising:
causing an elongated body to be inserted into a body cavity or vessel, said elongated body configured to carry ablative energy from an ablative energy source associated with a proximal end to a distal end;
causing said ablative energy source to introduce said ablative energy into said elongated body at said proximal end; and
causing a distal tip located at said distal end to deliver said ablative energy in a direction substantially lateral to a longitudinal axis of said elongated body.

16. The method as recited in claim 15 wherein said elongated body is a catheter.

17. The method as recited in claim 15 wherein said elongated body is a guidewire.

18. The method as recited in claim 15 wherein said elongated body is associated with at least one optical fiber and said ablative energy is a laser light.

19. The method as recited in claim 18 wherein said distal tip has an associated glass element with an angular surface to reflect said ablative energy in a direction substantially lateral to said longitudinal axis of said elongated body.

20. The method as recited in claim 19 wherein said glass element is fitted within a collar and said ablative energy is reflected by an angular surface thru a port.

21. The method as recited in claim 15 wherein said elongated body is associated with a plurality of optical fibers.

22. The method as recited in claim 21 wherein said plurality of optical fibers terminate at said distal tip and each is polished at an acute angle and oriented to propagate said ablative energy in a direction substantially lateral to said longitudinal axis of said elongated body.

23. The method as recited in claim 15 wherein said ablative energy is provided by radio frequency (RF) and said elongated body has an associated wound coil terminating at an electrode in said distal tip, wherein said ablative energy is directed substantially lateral to said longitudinal axis of said elongated body through an RF ablative port.

24. The method as recited in claim 23 wherein said coil is made of stainless steel and said electrode is an alloy of either platinum or cadmium.

25. The method as recited in claim 15 further comprising a balloon capable of being inflated by an infusion of liquid injected via a lumen within said elongated body, said balloon located proximate said distal tip.

26. The method as recited in claim 15 further comprising a radiopaque marker band proximate said distal tip.

27. The method as recited in claim 15 further comprising an imaging component proximate said distal tip.

28. The method as recited in claim 27 wherein said imaging component is an intravascular ultrasound device.
